(19) United States
(12) Patent Application Publication
(10) Pub. No.: US 2004/0219620 A1
(43) Pub. Date: Nov. 4, 2004

(54) Chromogenic substrate with a pH indicator dye

(76) Inventor: Brent A. Mayer, Lexington, KY (US)

Correspondence Address: MCLEOD & MOYNE, P.C., 2190 COMMONS PARKWAY, OKEMOS, MI, 48864, US

(21) Appl. No.: 10/426,169

(22) Filed: Apr. 29, 2003

(51) Int. Cl. G01N 33/53

(52) U.S. Cl. 435/7.92

(57) ABSTRACT

A composition for an enzyme-linked assay is described which comprises a chromogenic substrate that produces a detectable color with a maximum absorbance at one wavelength in reactions of the assay which contain the enzyme and a pH indicator dye that produces a detectable color with a maximum absorbance at a second wavelength when the pH of the reactions of the assay is changed to terminate the reactions. The chromogenic substrate enables colorimetric detection of the bindable substance in positive reactions of the assay and the pH indicator dye enables negative reactions of the assay to be calorimetrically distinguished from false negative reactions. Preferably, the composition is used in enzyme-linked immunoassays such as ELISAs. Most preferably, the enzyme is a peroxidase. In a further preferred embodiment, the chromogenic substrate is 3,3′,5,5′-tetramethylbenzidine (TMB) and the pH indicator dye is preferably selected from the group consisting of cresol red and m-cresol purple.

### CROSS-REFERENCE TO RELATED APPLICATION

Not applicable.

### STATEMENT REGARDING FEDERALLY SPONSORED RESEARCH OR DEVELOPMENT

Not applicable.

### REFERENCE TO A “COMPUTER LISTING APPENDIX SUBMITTED ON A COMPACT DISC”

Not Applicable.

### BACKGROUND OF THE INVENTION

(1) Field of the Invention

The present invention relates to a composition for an enzyme-linked assay which comprises a chromogenic substrate that produces a detectable color with a maximum absorbance at one wavelength in reactions of the assay which contain the enzyme and a pH indicator dye that produces a detectable color with a maximum absorbance at a second wavelength when the pH of the reactions of the assay is changed to terminate the reactions. The chromogenic substrate enables calorimetric detection of a bindable substance in positive reactions of the assay and the pH indicator dye enables negative reactions of the assay to be calorimetrically distinguished from false negative reactions. Preferably, the composition is used in enzyme-linked immunoassays such as ELISAs. Most preferably, the enzyme is a peroxidase. In a further preferred embodiment, the chromogenic substrate is 3,3′,5,5′-tetramethylbenzidine (TMB) and the pH indicator dye is preferably selected from the group consisting of cresol red and m-cresol purple.

(2) Description of Related Art

Enzyme-linked immunosorbent assays (ELISA) are the most commonly used immunoassays for a wide variety of applications in diagnostics, research, food testing, process quality assurance and quality control, and environmental testing. ELISAs utilize a label enzyme and a substrate for the enzyme to produce a detectable signal for quantitating antigens, haptens, or antibodies. The sensitivity of ELISAs depends on the particular label enzyme and substrate combination used. Horseradish peroxidase (HRP) has been found to be well suited for use as a label enzyme in ELISAs because it is highly specific, sensitive, and very stable in catalyzing chromogenic, luminescence, and fluorescence reactions.

To increase sensitivity of peroxidase-linked ELISAs, colorless or slightly colorless chromogenic substrates are used. These chromogenic substrates include 3,3′,5,5′-tetramethylbenzidine (TMB), ortho-phenylenediamine dihydrochloride (OPD), and 2,2′-azinobis [3-ethyl-benzothiazoline-6-sulfonic acid] diammonium salt (ABTS). U.S. Pat. No. 4,503,143 to Gerber et al. discloses ELISA and other immunoassays which use TMB as the chromogenic substrate. U.S. Pat. No. 5,206,150 to Tai discloses water-based TMB solutions. Colorless chromogenic substrates reduce background signal which increases the overall sensitivity of the assay. However, a problem associated with using colorless chromogenic substrates is that during dispensing of a colorless substrate into a plurality of transparent tubes or wells of an ELISA plate, it is difficult to determine immediately by visual examination whether the chromogenic substrate had been added to a particular tube or well or not. As a consequence, in some cases the chromogenic substrate may not be added to some tubes or wells or added twice to some tubes or wells.

A second problem associated with using colorless chromogenic substrates is determining whether a negative reaction is negative because the sample for the reaction did not contain the analyte being tested for or is negative because one or more reagents necessary for the reaction had been omitted, for example the colorless chromogenic substrate. The second problem is particularly acute in peroxidase-linked ELISAs which include an acid stop following oxidation of the chromogenic substrate. Because it is not possible to distinguish these false negatives due to omitted sample or acid stop from actual negatives due to absence of the analyte in the sample or other false negatives caused by some other failure in the reaction such as omitted sample or degraded analyte in a sample, the number of negative reactions in an assay is artificially elevated.

A solution to the first problem was disclosed in U.S. Pat. No. 6,221,624 B1 to Lihme and Wikborg which describes a pre-stained composition comprising TMB and a visible dye such as phloxine B, m-cresol purple, or the like. The dye is used at a concentration which imparts a color to a solution containing the composition which is visible to the eye. The colored solution enables the user to visually determine that the chromogenic substrate is or has been added to a tube or well of an ELISA plate during pipetting. The dye is preferably selected so as to have no absorbance in the absorbance range under the conditions where the reaction product of the reaction between the chromogenic substrate and the enzyme is to be detected and to have substantially no influence on the enzyme-chromogenic substrate reaction. The preferred dye is phloxine B which is used at a concentration which provides a visible when added to a peroxidase reaction. Most peroxidase-based immunoassays use an acid stop to end peroxidase reactions because the acid stop stabilizes the oxidized product thereby increasing sensitivity of the assay. Phloxine B is rendered colorless when an acid stop is added to the reaction. Therefore, the above composition comprising phloxine B or similar dyes does not enable the user after the reactions have been completed to determine whether a negative reaction is an actual negative caused by an absence of analyte in the sample or a false negative caused by an omission of the chromogenic composition.

The second problem has not been addressed. Therefore, a need remains for a method that will enable actual negative reactions (absence of analyte in the sample) to be distinguished from false negatives caused by omission of particular reaction reagents in peroxidase-linked immunoassays such as ELISAs whilst not interfering with positive reactions.

### SUMMARY OF THE INVENTION

The present invention provides a composition which comprises a chromogenic substrate with an absorbance at one wavelength and a pH indicator dye with an absorbance at a second wavelength. The composition enables both detection of a bindable substance and confirmation of actual negative reactions in enzyme-linked assays, particularly enzyme-linked immunoassays such as ELISAs. In particular, the chromogenic substrate enables calorimetric detection of the bindable substance in positive reactions of the assay and the pH indicator dye enables actual negative reactions of the assay to be calorimetrically distinguished from false negative reactions. The following illustrates several of the preferred embodiments of the present invention.

The present invention provides a method for detecting an analyte in a peroxidase-based enzyme-linked immunosorbent assay (ELISA) and distinguishing a negative reaction from a false negative reaction in the ELISA, which comprises (a) providing a composition comprising a mixture of a chromogenic substrate which is oxidizable by peroxide in a reaction for the ELISA catalyzed by the peroxidase of the ELISA to form a first color with an absorbance at a first wavelength, a pH indicator dye which forms a second color which is detectable by eye and has an absorbance at a second wavelength when a stop solution is added to the composition in the reaction vessel, and a peroxide; (b) adding an aliquot of the composition to each of the reaction vessels for the ELISA to form a reaction mixture; (c) incubating the reaction mixture for a time sufficient to oxidize the chromogenic substrate to the first color; (d) adding the stop solution to the reaction mixture to stop the reaction and to generate the second color; and (e) measuring spectrographically absorbances at the first and second wavelengths, wherein absorbance at the first wavelength indicates the detection of the analyte, absorbance at only the second wavelength indicates the negative reaction, and absence of absorbance at the first and second wavelengths indicates the false negative reaction or detecting the second color by eye wherein the second color indicates that the reaction mixture contains the composition and the stop solution.

The present invention further provides an enzyme immunoassay for calorimetric detection of an analyte of a type wherein a quantity of a first immunologic reagent such as an antibody, Fab fragment, Fv fragment, single-chain Fv polypeptide, or other antibody derivative is adsorbed to a solid support in a reaction vessel; a conjugate is formed between a second immunologic reagent and a peroxidase; the conjugate is admixed with a sample to be tested for the analyte, the analyte binds to the first immunologic reagent and to the conjugate to form an immunologic complex in solid phase; and the quantity of the analyte is determined by measuring the reaction of the immunologic complex with a chromogenic substrate oxidizable by peroxide and the peroxidase, wherein the improvement comprises (a) providing a liquid solution comprising the chromogenic substrate, peroxide, and a pH indicator dye, wherein the chromogenic substrate is oxidizable to a first color with an absorbance at a first wavelength and the pH indicator dye produces a second color which is detectable by eye and which has an absorbance at a second wavelength when a stop solution is added to the reaction vessel containing the liquid solution; (b) mixing the liquid solution with the immunologic complex in the reaction vessel to form a reaction mixture which oxidizes the chromogenic substrate; (c) adding the stop solution to the reaction mixture in the reaction vessel to produce the first and second colors; and, (d) measuring spectrographically the absorbances at the first and second wavelengths, wherein absorbance at the first wavelength indicates detection of the analyte, absorbance at the second wavelength indicates a negative reaction, and an absence of absorbance at the first or second wavelength indicates a false negative reaction or detecting the second color by eye wherein the second color indicates that the reaction mixture contains the liquid solution and the stop solution.

The present invention further provides an enzyme immunoassay for calorimetric detection of an antibody of a type wherein a quantity of an analyte is adsorbed to a solid support in a reaction vessel; a conjugate is formed between an immunologic reagent such as an antibody, Fab fragment, Fv fragment, single-chain Fv polypeptide, or other antibody derivative and a peroxidase the conjugate is admixed with a sample to be tested for the antibody, the antibody binds to the analyte and to the conjugate to form an immunologic complex in solid phase; and the quantity of the antibody is determined by measuring the reaction of the immunologic complex with a chromogenic substrate oxidizable by peroxide and the peroxidase, wherein the improvement comprises (a) providing a liquid solution comprising the chromogenic substrate, peroxide, and a pH indicator dye, wherein the chromogenic substrate is oxidizable to a first color with an absorbance at a first wavelength and the pH indicator dye produces a second color which is detectable by eye and which has an absorbance at a second wavelength when a stop solution is added to the reaction vessel containing the liquid solution; (b) mixing the liquid solution with the immunologic complex in the reaction vessel to form a reaction mixture which oxidizes the chromogenic substrate; (c) adding the stop solution to the reaction mixture in the reaction vessel to produce the first and second colors; and, (d) measuring spectrographically the absorbances at the first and second wavelengths, wherein absorbance at the first wavelength indicates detection of the analyte, absorbance at the second wavelength indicates a negative reaction, and an absence of absorbance at the first or second wavelength indicates a false negative reaction or detecting the second color by eye wherein the second color indicates that the reaction mixture contains the liquid solution and the stop solution.

Preferably, in the above methods, the pH indicator is colorless or has a color which is essentially not detectable by eye when the liquid solution is applied to the reaction vessel for the immunoassay but produces the second color which is detectable by eye and which has the absorbance at the second wavelength when the stop solution is added to the reaction vessel containing the liquid solution.

In a further embodiment of the above methods, the stop solution is an acid and the chromogenic substrate is selected from the group consisting of ortho-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), and 3,3′,5,5′-tetramethylbenzidine (TMB). In a further embodiment, the stop solution is an acid and the chromogenic substrate is 2,2′-azinobis-(3-ethyl-benzothiazoline-6-sulfonic acid) (ABTS).

In a further still embodiment of the above methods, the pH indicator dye is selected from the group consisting of cresol red, m-cresol purple, benzopurpurin 4B, metanil yellow, 4-phenylazodiphenylamine, malachite green, orange IV, 2,2′,2″,4,4″-pentamethoxytriphenyl carbinol, and combinations thereof. In a preferred embodiment, the pH indicator dye is m-cresol purple.

In a further still embodiment of the above methods, the stop solution is a base and the chromogenic substrate is 5-aminosalicylic acid (5AS) and in a further still embodiment, the pH indicator dye is selected from the group consisting of phenolphthalein, thymolphthalein, alizarin Yellow R, indigo carmine, and combinations thereof.

In further embodiments of the above methods, the ELISA is a direct immunoassay, an indirect immunoassay, or a competitive immunoassay.

The present invention further provides a method for detecting an analyte using 3,3′,5,5′-tetramethylbenzidine (TMB) as a chromogenic substrate in an enzyme-linked immunosorbent assay (ELISA) and distinguishing a negative reaction from a false negative reaction in the ELISA, which comprises (a) providing a composition comprising a mixture of the TMB which is oxidizable by peroxide in a reaction for the ELISA catalyzed by the peroxidase of the ELISA to form a first color with an absorbance at a first wavelength, a pH indicator dye which is colorless or has a color which is essentially not detectable by eye when the composition is applied to a reaction vessel for the ELISA but which forms a second color which is detectable by eye and has an absorbance at a second wavelength when a stop solution is added to the composition in the reaction vessel, and a peroxide; (b) adding an aliquot of the composition to each of the reaction vessels for the ELISA to form a reaction mixture; (c) incubating the reaction mixture for a time sufficient to oxidize the TMB to the first color; (d) adding the stop solution to the reaction mixture to stop the reaction, which produces the second color and further oxidizes the TMB to the first color; and (e) measuring spectrographically absorbances at the first and second wavelengths, wherein absorbance at the first wavelength indicates the detection of the analyte, absorbance at only the second wavelength indicates the negative reaction, and absence of absorbance at the first and second wavelengths indicates the false negative reaction or detecting the second color by eye wherein the second color indicates that the reaction mixture contains the composition and the stop solution.

The present invention further provides an enzyme immunoassay for calorimetric detection of an analyte of a type wherein a quantity of a first antibody is adsorbed to a solid support in a reaction vessel; a conjugate is formed between an immunologic reagent and a peroxidase; the conjugate is admixed with a sample to be tested for the analyte, the analyte binds to the first antibody and to the conjugate to form an immunologic complex in solid phase; and the quantity of the analyte is determined by measuring the reaction of the immunologic complex with 3,3′,5,5′-tetramethylbenzidine (TMB) which is oxidizable by peroxide and the peroxidase, wherein the improvement comprises (a) providing a liquid solution comprising the TMB, peroxide, and a pH indicator dye, wherein the TMB is oxidizable to a first color with an absorbance at a first wavelength and the pH indicator dye is colorless or has a color which is essentially not detectable by eye when the liquid solution is applied to the reaction vessel for the immunoassay but which produces a second color which is detectable by eye and which has an absorbance at a second wavelength when a stop solution is added to the reaction vessel containing the liquid solution; (b) mixing the liquid solution with the immunologic complex in the reaction vessel to form a reaction mixture which oxidizes the TMB; (c) adding the stop solution to the reaction mixture in the reaction vessel to produce the first and second colors; and, (d) measuring spectrographically the absorbances at the first and second wavelengths, wherein absorbance at the first wavelength indicates detection of the analyte, absorbance at the second wavelength indicates a negative reaction, and an absence of absorbance at the first or second wavelength indicates a false negative reaction or detecting the second color by eye wherein the second color indicates that the reaction mixture contains the liquid solution and the stop solution.

The present invention further provides an enzyme immunoassay for calorimetric detection of an antibody of a type wherein a quantity of an analyte is adsorbed to a solid support in a reaction vessel; a conjugate is formed between an immunologic reagent such as an antibody, Fab fragment, Fv fragment, single-chain Fv polypeptide, or other antibody derivative and a peroxidase; the conjugate is admixed with a sample to be tested for the antibody, the antibody binds to the analyte and to the conjugate to form an immunologic complex in solid phase; and the quantity of the antibody is determined by measuring the reaction of the immunologic complex with 3,3′,5, 5′-tetramethylbenzidine (TMB) which is oxidizable by peroxide and the peroxidase, wherein the improvement comprises (a) providing a liquid solution comprising the TMB, peroxide, and a pH indicator dye, wherein the TMB is oxidizable to a first color with an absorbance at a first wavelength and the pH indicator dye is colorless or has a color which is essentially not detectable by eye when the liquid solution is applied to the reaction vessel for the immunoassay but which produces a second color which is detectable by eye and which has an absorbance at a second wavelength when a stop solution is added to the reaction vessel containing the liquid solution; (b) mixing the liquid solution with the immunologic complex in the reaction vessel to form a reaction mixture which oxidizes the TMB; (c) adding the stop solution to the reaction mixture in the reaction vessel to produce the first and second colors; and, (d) measuring spectrographically the absorbances at the first and second wavelengths, wherein absorbance at the first wavelength indicates detection of the analyte, absorbance at the second wavelength indicates a negative reaction, and an absence of absorbance at the first or second wavelength indicates a false negative reaction or detecting the second color by eye wherein the second color indicates that the reaction mixture contains the liquid solution and the stop solution.

In a further embodiment of the method, the pH indicator dye is selected from the group consisting of cresol red, m-cresol purple, benzopurpurin 4B, metanil yellow, 4-phenylazodiphenylamine, malachite green, orange IV, 2,2′,2″,4,4″-pentamethoxytriphenyl carbinol, and combinations thereof. In a preferred embodiment, the pH indicator dye is m-cresol purple. In a further still embodiment, the first wavelength is about 450 nm and the second wavelength is over 500 nm, and further still the acid is selected from the group consisting of HCL, H₂SO₄, and H₂PO₄.

In further embodiments of the method, the ELISA is a direct immunoassay, an indirect immunoassay, or a competitive immunoassay.

The present invention further provides a kit for detecting an analyte in an enzyme-linked assay and distinguishing a negative reaction from a false negative reaction in the assay, which comprises one or more first containers each of which contains a mixture of a chromogenic substrate, a pH indicator dye suitable for use with the chromogenic substrate, which is colorless or has a color which is essentially not detectable by eye when the mixture is applied to a reaction vessel for the enzyme-linked assay but which produces a color detectable by eye when an acid or base stop solution is added to the mixture in the reaction vessel, and a peroxide.

The present invention further provides a kit for an enzyme-linked immunosorbent assay (ELISA) for detecting an analyte and distinguishing a negative reaction from a false negative reaction in the ELISA, which comprises one or more first containers each of which contains a mixture of a chromogenic substrate, a pH indicator dye suitable for use with the chromogenic substrate, which is colorless or has a color which is essentially not detectable by eye when the mixture is applied to a reaction vessel for the ELISA but which produces a color detectable by eye when an acid or base stop solution is added to the mixture, and a peroxide.

In a further embodiment of the above kits, the first container contains a mixture of a chromogenic substrate and a pH indicator dye and the peroxide is contained in a second container. In a further still embodiment, the kit further includes a third container containing an acid or base stop.

In a further still embodiments of the above kits, the chromogenic substrate is selected from the group consisting of ortho-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), and 3,3′,5,5′-tetramethylbenzidine (TMB).

In a further still embodiments of the above kits, the pH indicator dye is selected from the group consisting of cresol red, m-cresol purple, benzopurpurin 4B, metanil yellow, 4-phenylazodiphenylamine, malachite green, quinaldine red, orange IV, 2,2′,2″, 4,4″-pentamethoxytriphenyl carbinol, and combinations thereof.

In a further still embodiments of the above kits, the chromogenic substrate is 5-aminosalicylic acid (5AS) and further still, the pH indicator dye is selected from the group consisting of phenolphthalein, thymolphthalein, alizarin yellow R, indigo carmine, and combinations thereof.

In a further embodiments of the above kits, the pH indicator dye is m-cresol purple and the chromogenic substrate is selected from the group consisting of 3,3′,5,5′-tetramethylbenzidine (TMB) and 2,2′-azinobis-(3-ethyl-benzothiazoline-6-sulfonic acid) (ABTS).

The present invention further provides a kit for an enzyme-linked immunosorbent assay (ELISA) for detecting an analyte, which comprises one or more first containers each of which contains a mixture of 3,3′,5,5′-tetramethylbenzidine (TMB), a pH indicator dye suitable for use with the chromogenic substrate, which is colorless or has a color which is essentially not detectable by eye when the mixture is applied to a reaction vessel for the ELISA but which produces a color detectable by eye when an acid or base stop solution is added to the mixture in the reaction vessel, and a peroxide. Preferably, the pH indicator is m-cresol purple.

In a further embodiment of the kit, the first container contains a mixture of the TMB and a pH indicator dye and the peroxide is contained in a second container. In a further embodiment, the kit further includes a third container containing an acid or base stop.

The present invention further provides a composition for use in an enzyme-linked immunosorbent assay (ELISA) and which enables a negative reaction to be distinguished from a false negative reaction in the ELISA, which comprises in a mixture (a) a chromogenic substrate which is oxidizable by a peroxide generated in the ELISA in a reaction mixture to which a stop solution is then added to stop the reaction to form a first color; and (b) a pH indicator dye which is colorless or has a color which is essentially not detectable by eye when the composition is applied to a reaction vessel for the ELISA but which produces a second color detectable by eye and which has an absorbance at a second wavelength when an acid or base stop solution is added to the composition in the reaction vessel.

In a further embodiment of the composition, the chromogenic substrate is selected from the group consisting of ortho-phenylenediamine (OPD), 2,2′-azinobis-(3-ethyl-benzothiazoline-6-sulfonic acid) (ABTS), diaminobenzidine (DAB), and 3,3′dimethyloxybenzidine (ortho-dianisidine or ODN).

In a further still embodiment of the composition, the pH indicator dye is selected from the group consisting of cresol red, m-cresol purple, benzopurpurin 4B, metanil yellow, 4-phenylazodiphenylamine, malachite green, quinaldine red, orange IV, 2,2′,2″,4,4″-pentamethoxytriphenyl carbinol, and combinations thereof.

In a further still embodiment of the composition, the chromogenic substrate is 5-aminosalicylic acid (5AS) and further still, the pH indicator dye is selected from the group consisting of phenolphthalein, thymolphthalein, alizarin yellow R, indigo carmine, and combinations thereof.

In a further still embodiment of the composition, the pH indicator dye is m-cresol purple and the chromogenic substrate is 2,2′-azinobis-(3-ethyl-benzothiazoline-6-sulfonic acid) (ABTS).

The present invention further provides a composition for use in an enzyme-linked immunosorbent assay (ELISA) and which enables a negative reaction to be distinguished from a false negative reaction in the ELISA, which comprises in an aqueous mixture (a) 3,3′,5,5′-tetramethylbenzidine (TMB) which is oxidizable by a peroxide generated in the ELISA in a reaction mixture to which a stop solution is then added to stop the reaction to form a first color; and (b) an indicator dye which is colorless or has a color which is essentially not detectable by eye when the composition is applied to a reaction vessel for the ELISA but which produces a color detectable by eye and which has an absorbance at a second wavelength when an acid or base stop solution is added to the composition in the reaction vessel.

In a further embodiment of the composition, the pH indicator dye is selected from the group consisting of cresol red, benzopurpurin 4B, metanil yellow, 4-phenylazodiphenylamine, malachite green, quinaldine red, orange IV, 2,2′, 2″,4,4″-pentamethoxytriphenyl carbinol, and combinations thereof.

In a further still embodiment of the composition, the pH indicator dye is m-cresol purple.

In a further embodiment, the present invention provides a method for detecting an analyte in a enzyme-linked assay and distinguishing a negative reaction from a false negative reaction in the assay, which comprises (a) providing a composition comprising a mixture of a chromogenic substrate selected from the group consisting of ortho-phenylenediamine (OPD), 2,2′-azinobis-(3-ethyl-benzothiazoline-6-sulfonic acid) (ABTS), diaminobenzidine (DAB), and 3,3′dimethyloxybenzidine (ortho-dianisidine or ODN) which is converted by an enzyme in a reaction vessel for the assay to a first color which has an absorbance at a first wavelength and a pH indicator dye which produces a second color detectable by eye and which has an absorbance at a second wavelength when an acid or base stop solution is added to the composition in the reaction vessel; (b) adding an aliquot of the composition to the reaction vessels for the enzyme-linked assay to form a reaction mixture; (c) incubating the reaction mixture for a time sufficient to produce the first color; (d) adding the stop solution to the reaction mixture to stop the reaction and to generate the second color; and (e) measuring spectrographically absorbances at the first and second wavelengths, wherein absorbance at the first wavelength indicates the detection of the analyte, absorbance at only the second wavelength indicates the negative reaction, and absence of absorbance at the first and second wavelengths indicates the false negative reaction or detecting the second color by eye wherein the second color indicates that the reaction mixture contains the composition and the stop solution.

In a further embodiment of the method, the enzyme is an alkaline phosphatase, the chromogenic substrate is p-nitrophenyl phosphate, and the stop solution is a base. In a further still embodiment, the pH indicator dye is selected from the group consisting of alizarin Yellow R, indigo carmine, cresol red, m-cresol purple, benzopurpurin 4B, metanil yellow, 4-phenylazodiphenylamine, malachite green, orange IV, 2,2′,2″,4,4″-pentamethoxytriphenyl carbinol, and combinations thereof.

In further still embodiments, the assay is a direct immunoassay, an indirect immunoassay, or a competitive immunoassay. In a further embodiment, the assay is an enzyme-linked immunosorbent assay (ELISA).

The present invention further provides a kit for detecting an analyte in an enzyme-linked assay and distinguishing a negative reaction from a false negative reaction in the assay, which comprises one or more containers each of which contains a mixture of a chromogenic substrate selected from the group consisting of ortho-phenylenediamine (OPD), 2,2′-azinobis-(3-ethyl-benzothiazoline-6-sulfonic acid) (ABTS), diaminobenzidine (DAB), and 3,3′dimethyloxybenzidine (ortho-dianisidine or ODN) which is converted by an enzyme in a reaction vessel for the assay to a first color and a pH indicator dye suitable for use with the chromogenic substrate which produces a second color detectable by eye when an acid or base stop solution is added to the mixture in the reaction vessel.

In a further embodiment of the kit, the chromogenic substrate is p-nitrophenyl phosphate. In a further still embodiment, the pH indicator dye is selected from the group consisting of alizarin Yellow R, indigo carmine, cresol red, m-cresol purple, benzopurpurin 4B, metanil yellow, 4-phenylazodiphenylamine, malachite green, orange IV, 2,2′,2″,4,4″-pentamethoxytriphenyl carbinol, and combinations thereof.

In a further embodiment of the kit, the assay is an enzyme-linked immunosorbent assay (ELISA).

The present invention further provides a composition for use in an enzyme-linked assay and which enables a negative reaction to be distinguished from a false negative reaction in the assay, which comprises in a mixture (a) a chromogenic substrate selected from the group consisting of ortho-phenylenediamine (OPD), 2,2′-azinobis-(3-ethyl-benzothiazoline-6-sulfonic acid) (ABTS), diaminobenzidine (DAB), and 3,3′dimethyloxybenzidine (ortho-dianisidine or ODN) which is converted by an enzyme in a reaction vessel for the assay to a first color; and (b) a pH indicator dye which is colorless or has a color which is essentially not detectable by eye when the composition is applied to a reaction vessel for the ELISA but which produces a second color detectable by eye and which has an absorbance at a second wavelength when an acid or base stop solution is added to the composition in the reaction vessel.

In a further embodiment of the composition, the chromogenic substrate is p-nitrophenyl phosphate. In a further still embodiment, the pH indicator dye is selected from the group consisting of alizarin Yellow R, indigo carmine, cresol red, m-cresol purple, benzopurpurin 4B, metanil yellow, 4-phenylazodiphenylamine, malachite green, orange IV, 2,2′,2″,4,4″-pentamethoxytriphenyl carbinol, and combinations thereof.

In a further embodiment of the composition, the assay is an enzyme-linked immunosorbent assay (ELISA).

### OBJECTS

It is an object of the present invention to provide a method for distinguishing negatives from false negatives in assays which does not interfere with positive reactions.

It is a further object of the present invention to provide a method for distinguishing negatives from false negatives in peroxide-linked assays which does not interfere with positive reactions.

It is a further object of the present invention to provide a method for distinguishing negatives from false negatives in ELISAs which does not interfere with positive reactions.

It is a further object of the present invention to provide a method for distinguishing negatives from false negatives in peroxide-linked ELISAs which does not interfere with positive reactions.

These and other objects of the present invention will become increasingly apparent with reference to the following drawings and preferred embodiments.

### DESCRIPTION OF THE DRAWINGS

FIG. 1 shows comparative absorbance spectra for the composition comprising TMB and m-cresol purple with and without horseradish peroxidase (HRP). The black line (-) is the absorbance spectrum for a sample containing the composition. The green line (-·-) shows the absorbance spectrum for a sample containing the composition and acid stop. The blue line (-×-) shows the absorbance spectrum for a sample containing the composition incubated with HRP for 15 minutes. The red line (-|-) shows the absorbance spectrum for a sample containing the composition incubated with HRP for 15 minutes followed by adding the acid stop. All spectra were measured against water on a Shimadzu UV-1601 spectrophotometer.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “chromogenic substrate” includes chemicals which can participate in particular enzymatic reactions as either donors or acceptors for the reaction and which changes color during the reaction. For example, peroxidase converts hydrogen peroxide to water. It does so by obtaining two hydrogens from a donor molecule. When the donor molecule is a chromogenic substrate, the oxidation of the chromogenic substrate causes the substrate to change to a detectable color. For example, 3,3′,5,5′-tetramethylbenzidine (TMB) is colorless in the reduced state but blue in the oxidized state or yellow in the diamine state. In the case of alkaline phosphatase reactions, the chromogenic substrate is converted from a colorless form to a visible color. For example, when the substrate is p-nitrophenyl phosphate (pNPP), the alkaline phosphatase converts it to p-nitrophenoxide (benzenoid form) which is colorless. However, at alkaline pH, the benezoid form is converted to a quinonoid form which is yellow.

The term “pH indicator” as used herein includes chemicals that take on different colors depending upon the concentration of hydronium or hydroxide ion present in an aqueous solution. Many pH indicators change from one color to another or from colorless to a color over a particular pH range. This is called the pH indicator range and this range varies from pH indicator to pH indicator. The term “pH indicator dye” as used herein further includes any dye which changes color or produces a color upon a change in pH but which is not commonly used as an indicator for detecting pH changes per se.

The phrase “has a color which is essentially not detectable by eye when applied to a reaction vessel for an assay” means that a composition comprising the pH indicator dye and chromogenic substrate has a color which an operator of the assay would not readily perceive in the normal course of performing the assay. A color which is not readily perceptible is a color which is too faint or of insufficient intensity to be detected by the unaided human eye, particularly at the volume which is applied to the reaction vessel and particularly when the composition is at a pH between about 3 and 6.5. In general, as the volume of a solution with a detectable color decreases, the less detectable or perceptible to the eye becomes the color of the solution. For example, while the chromogenic substrate and pH indicator dye compositions of the present invention might have a color which in a large volume might be perceptible or detectable by eye, the volume applied to a reaction vessel for an assay is not sufficient to enable the color to be readily perceptible or detectable by the operator's eye.

The phrase “normal course of performing a reaction in an assay” means performing an assay using a volume of the chromogenic substrate and pH indicator dye which is routinely used for the type of assay. For example, many ELISAs use between about 50 to 500 μL of chromogenic substrate and peroxide solution per reaction well. Thus, in the present invention, the well would contain about 50 to 500 μL of the chromogenic substrate, pH indicator dye, and peroxide solution per reaction well.

The term “ELISA” refers to enzyme-linked immunosorbent assay and includes direct, indirect, and competitive ELISAs. The basic principle of an ELISA is to use an enzyme to detect the binding of antigen and antibody. The enzyme converts a colorless chromogenic substrate to a colored product, indicating the presence of antigen-antibody binding. An ELISA can be used to detect either the presence of particular antibodies or antigens in a sample. A direct ELISA is used primarily to determine the amount of antigen in a sample. In a direct ELISA, antibody for a particular antigen is immobilized in the reaction vessel or well of a microtiter plate or other solid support. The immobilized antibody binds the antigen in a sample and the bound antigen is detected by an enzyme-labeled antibody specific for the antigen. An indirect ELISA is used primarily to determine the strength or amount of an antibody response to a particular antigen in a sample such as serum from an animal. In an indirect ELISA, an antigen is immobilized in the reaction vessel or well of a microtiter plate or other solid support. The immobilized antigen binds antibody in a sample which is specific for the antigen and the bound antibody is detected by an enzyme-labeled antibody specific for the bound antibody. For both direct and indirect ELISAs, the amount of colored product is directly proportional to the amount of antigen or antibody in the sample. A competitive ELISA is a variation where either (a) the amount of antibody in a sample is determined in a competition reaction between antibody in the sample and enzyme-labeled antibody for an antigen immobilized in the reaction vessel or well of a microtiter plate or other solid support or (b) the amount of antigen in a sample is determined in a competition reaction between the antigen in the sample and enzyme-labeled antigen for antibody specific for the antigen immobilized in the reaction vessel or well of a microtiter plate or other solid support. In a competitive ELISA, the amount of colored product is inversely proportional to the amount of antibody or antigen in the sample.

The term “immunoassay” includes all methods for detecting an antigen or antibody in a sample such as ELISAs and the like, and includes direct, indirect, and competitive immunoassays.

The term “analyte” includes both antigen (including haptens and lectins) and antibody. Whether the analyte is an antigen or an antibody depends on the type of immunoassay. For example, in immunoassays for detecting an antigen, the antigen is the analyte, whereas in immunoassays for detecting an antibody, the antibody is the analyte.

The terms “immunologic reagent” and “antibody” include whole antibodies and derivatives thereof. For example, the terms include Fab fragments, recombinant Fab polypeptides, Fv fragments, recombinant single-chain Fv polypeptides, and variations thereof. The terms also include both polyclonal antibodies and monoclonal antibodies and antibodies and derivatives thereof made by recombinant DNA methods.

The term “reaction vessel” includes test tube, well of a microtiter or tissue culture plate, capillary tube, cuvette, and the like.

The present invention provides a solution to the problem of whether a negative reaction in enzyme-linked assays such as ELISAs (direct, indirect, and competitive), immunoassays (direct, indirect, and competitive), hybridizations, or the like, is negative because the test sample did not contain the analyte being tested for or is negative because one or more reagents necessary for the reaction had been omitted by including in a ready-to-use composition for enzyme-linked assays a pH indicator dye which produces a detectable color or absorbance when a stop solution is added to the composition in the reaction vessel. In a preferred embodiment, the pH indicator dye is colorless or has a color which is not detectable by eye when the composition is applied to a reaction vessel for the assay but which produces the detectable color when the stop solution is added to the composition in the reaction vessel. The present invention is particularly useful for peroxidase-linked assays, particularly immunoassays such as ELISAs, and particularly is those assays which use an acid or base stop step to enhance sensitivity of the assay. In a preferred embodiment, the peroxidase is horseradish peroxidase (HRP). The present invention is also useful for alkaline phosphatase-linked assays, particularly in ELISAs which use p-nitrophenyl phosphate as the substrate.

In a typical peroxidase-linked immunoassay, the peroxidase is conjugated to a first member of a binding pair, for example an antibody or analyte. The peroxidase-binding pair conjugate is incubated with a sample which is suspected of containing the second member of the binding pair (analyte or antibody) in a reaction vessel. After a time sufficient to enable the peroxidase-binding pair conjugate to bind the second member of the binding pair to form a peroxidase-labeled complex, unbound material is removed and the peroxidase-labeled complex is detected by incubating the complex in a solution comprising a peroxide and a chromogenic substrate which is preferably in a water-soluble leuco form. The peroxidase catalyzes oxidation by the peroxide of the chromogenic substrate to a detectable color. For particular chromogenic substrates such as 3,3′,5,5′-tetramethylbenzidine (TMB) or ortho-phenylenediamine (OPD), the sensitivity of the reaction is enhanced by stopping the oxidation reaction with a stop solution containing an acid, which in the case of TMB is an increase of sensitivity of about 2 to 4 fold. A preferred peroxidase is HRP. HRP is most active between about pH 5 and pH 7.

In the present invention, the pH indicator dye is preferably selected to be colorless or have a color which is essentially undetectable by eye when a composition comprising a chromogenic substrate and the pH indicator dye is applied to a reaction vessel for the assay (the concentration of the pH indicator dye in the composition, the pH of the composition, and the volume of the composition together results in the color of the pH indicator dye being essentially undetectable by eye) but to produce a color which is detectable by eye and by absorbance when the pH of the composition is lowered to below the reaction pH by addition of an acid stop solution to the reaction vessel or raised to a pH above the reaction pH by addition of a base stop solution to the reaction vessel. However, in particular embodiments comprising particular chromogenic substrates, the pH indicator can have a color which is detectable by eye when a composition comprising a chromogenic substrate and the pH indicator dye is applied to a reaction vessel for the assay. Upon addition of the stop solution, the color can remain the same or the color can be changed to another color.

An additional requirement for the pH indicator dye is that the color or absorbance wavelength which is produced upon addition of the acid or base does not substantially interfere with or otherwise substantially affect detection of the absorbance wavelength produced by oxidation of the chromogenic substrate in positive reactions. In other words, the color produced by the pH indicator dye has an absorbance range which is outside of or does not substantially interfere with the absorbance range for the chromogenic substrate after acid or base has been added to the reaction. Preferably, the pH indicator dye is transparent in the absorbance range of the chromogenic substrate color produced prior to the addition of the acid or base. For example, TMB is oxidized to a blue color which is converted to a yellow upon addition of acid.

A further requirement for the pH indicator dye is that the pH indicator dye must be substantially non-interfering in the reaction between the chromogenic substrate and the enzyme.

In a particularly useful embodiment of the present invention, ready-to-use compositions are provided which comprise a mixture of the pH indicator dye, a chromogenic substrate, and a peroxide. Preferably, the composition further includes a buffer and other compounds which might stabilize the chromogenic substrate and/or pH indicator prior to or during the reaction or facilitate solubility of the chromogenic substrate in the reaction or during storage when the composition is provided as an aqueous solution. Most preferably, the compositions are liquid.

Suitable chromogenic substrates for peroxidase-linked assays include, but are not limited to, ortho-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), 5-aminosalicylic acid (5AS), and 3,3′,5,5′-tetraalkylbenzidine such as 3,3′,5,5′-tetramethylbenzidine (TMB). Other chromogenic substrates include various fluorescent and chemiluminescent compounds.

OPD is colorless but is oxidized in a peroxidase reaction to a yellow color with a maximum absorbance at 450 nm. The addition of acid to the reaction produces a stable orange color with an absorbance maximum at 490 nm. ABTS is oxidized in a peroxidase reaction to a blue-green color with a maximum absorbance at 405-410 nm. The addition of 1% SDS or acid to the reaction produces a stable blue-green color with an absorbance maximum at 405-410 nm. DAB is oxidized to an insoluble brown precipitate. As such, it is used primarily for immunohistochemical assays. OND is oxidized in a peroxidase reaction to a yellow-orange color with an absorbance maximum at 460 nm. The reaction can be stopped by the addition of an acid such as HCL and the reaction product read. 5AS is oxidized in a peroxidase reaction to a brown color with an absorbance maximum at 450 nm. The reaction can be stopped by the addition of NaOH and the reaction product read at 550 nm. TMB is colorless but is oxidized in a peroxidase reaction to a blue color with a maximum absorbance at 650 nm. The addition of acid to the reaction produces a stable yellow color with an absorbance maximum at 450 nm. Of the above, TMB is most preferred by those skilled in the art because it is non-hazardous and the most widely used of chromogenic substrates for peroxide-linked immunoassays such as ELISAs and the like. The present invention includes compositions comprising any one of the above chromogenic substrates in combination with a pH indicator dye and peroxide.

It is preferred that the chromogenic substrate be provided in an aqueous or water-base solution. Preferably, the aqueous solution is 100% water-based and does not include any organic solvents. It is further preferable that the solution be weakly buffered. In particular compositions, it may be necessary or desirable to include one or more solubility increasing agent(s) in order to facilitate the (permanent) dissolution of the chromogenic substrate. An example of a suitable solubility increasing agent is polyvinyl alcohol. Thus, in particular embodiments, the chromogenic substrate can comprise a solvent system comprising less than 5% (v/v) of organic solvents. It is further preferable that the chromogenic substrate be provided in mixture with a peroxide such as hydrogen peroxide or urea peroxide.

In a preferred embodiment, the chromogenic composition comprises TMB in a solution which is preferably aqueous or water-based. Examples of commercially available TMB chromogenic compositions which can be modified to include a pH indicator dye include, but are not limited to, K-BLUE SUBSTRATE, K-BLUE MAX SUBSTRATE, and the like available from Neogen Corp. Lansing, Mich.; TMB ONE, TMB PLUS, and the like available from Kem-En-Tec A/S, Copenhagen, Denmark; TMB substrate, TMB substrate with tracking dye, and the like available from BioFX Laboratories, Inc., Owning Mills, Md.; and, TMB Subtrate Kit available from Vector Laboratories, Inc. Burlingame, Calif. In a most preferred embodiment, the TMB chromogenic composition comprises a 100% aqueous or water-based solution which is the same as or similar to the TMB chromogenic solution disclosed in U.S. Pat. No. 5,206,150 to Tai. In a particularly preferred embodiment, the TMB chromogenic composition does not contain any organic solvents. It is further preferable that the solution be weakly buffered.

The pH indicator dye which is preferred for the present invention is colorless at a pH greater than about 2.8 or provides a color at a pH greater than about 2.8 but less than about pH 7.0 that is essentially not detectable by eye at the concentration or volume used in the reaction of an assay. Preferably, the pH indicator dye is water-soluble in at least the oxidized state or the reduced state. Preferably, the pH indicator dye is water-soluble in both states. It is particularly important that the pH indicator dye change color at a pH which is below or above the pH in which the chromogenic substrate oxidation or reduction reaction is performed and that the color, which is produced by the pH indicator dye when the reaction is stopped by addition of acid or base, is readily detectable by eye but substantially transparent in the absorbance range of the oxidized or reduced chromogenic substrate. However, it is preferable that the pH indicator also have an absorbance at a wavelength which does not interfere with the absorbance range of the oxidized or reduced chromogenic substrate. Thus, the color produced by the pH indicator dye is not only visible to the eye but is measurable at a wavelength other than the wavelength used to measure the oxidized or reduced chromogenic substrate. This is particularly useful for analyses wherein a detector is adapted to measure the absorbance of the oxidized or reduced chromogenic substrate and the absorbance of the oxidized or reduced pH indicator. Such detectors include ELISA readers.

Examples of pH indicator dyes which change color at an acid pH and are useful in the present invention include, but are not limited to, cresol red (yellow to red transition at pH 0.5-1.8), m-cresol purple (yellow to red transition at pH 1.2-2.8), 2,2′,2″,4,4′-pentamethoxytriphenyl carbinol (colorless to red transition at pH 1.2 to 3.2), and benzopurpurin 4B (red to violet transition at pH 2.2-4.2). Other acid pH indicator dyes which might be useful include but are not limited to, metanil yellow (yellow to red transition at pH 1.2-3.0), 4-phenylazodiphenylamine (yellow to red transition at pH 1.2-2.5), malachite green (yellow to blue-green transition at pH 0.2-1.8), quinaldine red (colorless to red transition at pH 1.0-2.2), orange IV (yellow to red transition at pH 1.4-2.8), thymol blue (red to yellow transition at pH 1.2 to 2.8), xylenol blue (yellow to red transition at pH 1.8 to 2.8), and combinations thereof.

Examples of pH indicator dyes which change color at an alkaline pH and are useful with chromogenic substrates such as 5AS include, but are not limited to, such dyes as phenolphthalein (colorless to red transition at pH 8.0 to 10), thymolphthalein (colorless to blue transition at pH 8.8 to 10.5), alizarin Yellow R (yellow to violet transition at pH 10 to 12), indigo carmine (blue to yellow transition at pH 11.4 to 13), m-cresol purple (yellow to purple transition at pH 7.4 to 9.0), cresol red (yellow or orange to purple transition at pH 7.0-8.8), thymol blue (yellow to blue transition at pH 8.0 to 9.6), xylenol blue (yellow to blue transition at pH 8.0 to 9.6), and combinations thereof.

The particular pH indicator dye used in an assay is that pH indicator dye which is suitable for use with the chromogenic substrate used in the assay. In other words, the pH indicator has an absorbance which does not interfere with the absorbance of the chromogenic substrate following the reaction and produces a color which is detectable by eye (visibly colored) after stopping the reaction with a stop solution but which is colorless or has a color which is essentially undetectable by eye when in the reaction vessel prior to adding the stop solution to the reaction. A preferred pH indicator dye for use in combination with TMB is m-cresol purple. The m-cresol purple is preferably used at a concentration which renders it essentially undetectable to the eye at the volume the TMB chromogenic solution is applied to a reaction vessel and at the pH of most TMB chromogenic solutions (most TMB chromogenic solutions are weakly buffered, usually in a buffer between about pH 3.8 and about pH 6.5) but which still enables it to be detectable by eye when the pH of the solution is reduced by addition of a acid stop solution. The ability to detect by eye the m-cresol purple in a composition containing the TMB in a reaction vessel prior to adding the acid stop solution is a function of the concentration of the m-cresol purple in the composition and the volume of the composition in the reaction vessel. Thus, the m-cresol purple is at a concentration in the composition which renders it visually undetectable when the composition is applied to a reaction vessel.

When an acid stop solution is added to the composition in the reaction vessel, the pH is reduced to below pH 2.8 and the m-cresol purple is oxidized to a rose-pink color. The rose-pink color is detectable by eye even when the m-cresol purple is at a concentration and the composition is at a volume which renders it essentially undetectable by eye prior to adding the acid stop solution. The oxidized m-cresol purple has a maximum absorbance at about 524-542 nm. The maximum absorbance does not interfere with the maximum absorbance of TMB (or OPD, ODN, or ABTS). Thus, either by visual observation or by measuring the absorbance at 524-542 nm, a user of the composition can readily determine whether a negative reaction was because the sample did not contain the analyte or the composition had been omitted from the reaction.

In a typical detection reaction for a peroxidase conjugate (for example, an HRP conjugate), the reaction vessel comprises in aqueous mixture the peroxidase conjugate and a composition comprising an oxidizable chromogenic substrate such as TMB, OPD, OND, 5AS, ABTS, or the like, and a pH indicator dye with an absorbance at an acid pH or basic pH different from the absorbance of the oxidized chromogenic substrate at the acid pH or basic pH. For example, oxidized TMB, OPD, and ABTS at an acid pH have a maximum absorbance at 450, 490, and 405 nm, respectively, and acid-oxidized m-cresol purple has a maximum absorbance at 530 nm. Preferably, the above composition further includes hydrogen or urea peroxide. It is further preferable, that the ratio of the chromogenic substrate to the pH indicator dye is between 10 to 1 and 1 to 10.

During the reaction, the chromogenic substrate is oxidized or reduced to a state which has a first color or maximum absorbance wavelength. The reaction is then stopped by the addition of a stop solution comprising an acid (hydrochloric acid (HCL), sulfuric acid (H₂SO₄), phosphoric acid (H₂PO₄), or the like) or a base such as sodium hydroxide (NaOH). The acid or base stops the above oxidation or reduction of the chromogenic substrate and for some substrates such as TMB or OPD, causes a further oxidation of the chromogenic substrate to a second color or maximum absorbance wavelength. It also causes oxidation or reduction of the pH indicator dye to a third color or maximum absorbance wavelength. For other chromogenic substrates such as ABTS or OND, the color of the oxidized or reduced product does not change upon addition of the acid. For 5AS, the reaction is stopped with a base such as NaOH which converts the oxidized chromogenic substrate to a product with an absorbance maximum at 550 nm. For particular formulations of TMB, the color of the TMB remains blue after the addition of the acid stop.

In a typical reaction, a positive reaction containing peroxidase conjugate is detected visually by eye or by absorbance at the maximum absorbance wavelength for the oxidized chromogenic substrate and a negative reaction which does not contain peroxidase conjugate is detected visually by eye or by absorbance at the maximum absorbance wavelength for the oxidized pH indicator. In the case of false negatives-reactions such as those which contain peroxidase conjugate but not the composition comprising the chromogenic substrate, the acid, or both, the reaction is colorless by eye and has no absorbance at either the first or second or third maximum absorbance wavelengths. Thus, reactions which have no absorbance at the first or second maximum absorbance wavelengths but have absorbance at the third maximum absorbance wavelength are scored as negatives whereas all reactions with no absorbance at the third maximum absorbance wavelength are scored as false negatives.

In an automated ELISA reader, the ELISA reader will correctly score reactions with a first (without acid) or second maximum absorbance wavelength (with acid) as positives and reactions with the third maximum absorbance wavelength as negatives. In the case of a reaction in which either the acid (or base) or the composition had been omitted, there is no first, second, or third maximum absorbance wavelength. An automated ELISA reader will correctly score the above reaction as a false negative. Thus, the composition of the present invention not only enables detection of positive reactions but also enables negative reactions to be distinguished from false negatives.

While the above compositions comprise a pH indicator dye which is colorless or have a color which is essentially not detectable by eye when the composition is applied to a reaction vessel for the assay, in the case of assays comprising OPD, OND, 5AS, ABTS and some assays comprising TMB, the pH indicator dye comprising the composition can have a color which is detectable by eye when the composition is applied to the reaction vessel. The pH indicator dye does not need to be colorless or have a color which is essentially not detectable by eye when the composition is applied to the reaction vessel. However, the pH indicator dye comprising the composition preferably forms a second color which is detectable by eye and has an absorbance at a second wavelength when a stop solution is added to the composition in the reaction vessel. The second wavelength cannot substantially interfere with detection of the first wavelength for the color of formed by the chromogenic substrate.

In a preferred detection reaction, the reaction comprises HRP conjugate in an aqueous composition comprising TMB as the chromogenic substrate and a pH indicator dye such as m-cresol purple. Preferably, the above composition further includes hydrogen or urea peroxide. It is further preferable that the composition is 100% water-based. For many formulations, the ratio of TMB to pH indicator dye is usually between about 10 to 1 and 1 to 10. In general, the chromogenic solution and reaction have a pH of about 3.8 to 6.5. When the pH indicator dye is m-cresol purple, the preferred amount of the m-cresol purple is less than about 10 μg m-cresol purple sodium salt per mL of TMB substrate, more preferably, less than about 2.2 μg/mL, more preferable still about 1.1 μg/mL. This amount of m-cresol purple in solution with the TMB substrate renders it essentially indistinguishable or undetectable by eye at the volume normally used for a reaction in an assay from solutions without the m-cresol purple prior to the addition of stop solution at the volume but still produces a distinguishable or detectable color after addition of the stop solution.

During the reaction, the TMB is oxidized to a state which is visually blue and which has an absorbance maximum at about 650 nm. Upon addition of a stop solution (preferably, a stop solution which comprises HCL at about 1 M or H₂SO₄ at about 0.2 M), the pH of the reaction is reduced. The reduced pH stops the reaction and further oxidizes TMB in the blue state to a diamine state. In the diamine state, the oxidized TMB is visually yellow and has a maximum absorbance at about 450 nm. Meanwhile, the m-cresol purple, which is essentially undetectable at pH 3.8 to 6.5 because of its low concentration in the composition (less than about 10 μg/mL) and the small reaction volume (in general, usually less than about 200 μL), is oxidized by the acid to a rose-pink (light red) which is visible to the eye and has a maximum absorbance at about 530-540 nm. Therefore, in a typical reaction, a positive reaction containing HRP conjugate can be detected visually by eye as a rose-pink or by absorbance at 450 nm and a negative reaction not containing HRP conjugate can be detected visually by eye as a rose-pink color or by measuring absorbance at about 520 to 565 nm, preferably at about 530 or 540 nm. In the case of false negatives-reactions such as those which contain HRP conjugate but in which the preferred composition, the acid stop, or both had been omitted, the reaction is colorless by eye and has no significant absorbance at 450 or 530 or 540 nm. Thus, reactions which have no absorbance at 450 nm but have absorbance at 530 or 540 nm are scored as negatives whereas all reactions with no absorbance at 530 or 540 nm are scored as false negatives.

In an automated ELISA reader, the ELISA reader will correctly score the yellow reactions as positives and the rose pink reactions as negatives. In the case of a reaction in which either the acid stop or the composition had been omitted, the color that is produced is blue or colorless, respectively. An automated ELISA reader will correctly score the above reactions as false negatives.

In a further embodiment of the above method using TMB as the chromogenic substrate, acid stops which stop the reaction but does not change the blue color of the reaction to yellow such as RED STOP (available from Neogen Corp., Lansing, Mich.) can be used. Thus, the absorbance of positive reactions remains blue and its absorbance read at 650 nm. The pH indicator dye is selected not to absorb in the 650 absorbance range. M-cresol purple is one such pH indicator dye which does not absorb at 650 nm.

The present invention further includes kits comprising the above compositions. The above compositions can be provided in liquid or solid form. When provided in solid form, the composition can be in capsule, tablet, or powder form. In one embodiment, the kit comprises one or more first containers each of which contains a mixture of a chromogenic substrate and a pH indicator dye suitable for use with the chromogenic substrate and one or more second containers each of which contains a peroxide such as hydrogen peroxide or urea peroxide. In a second embodiment, the kit comprises one or more containers each of which contains a mixture of a chromogenic substrate, a peroxide such as hydrogen peroxide or urea peroxide, and a pH indicator suitable for use with the chromogenic substrate. In further embodiments of the above kits, the kits further contain a container containing a stop solution.

The above kits are particularly useful for use in peroxidase-linked immunoassays such as ELISAs. In the above kits, it is preferable that the chromogenic substrate is selected from the group consisting of OPD, OND, ABTS, 5AS, and TMB. It is further preferable that the pH indicator dye for acid stops be selected from the group consisting of cresol red, m-cresol purple, benzopurpurin 4B, metanil yellow, 4-phenylazodiphenylamine, malachite green, quinaldine red, orange IV, 2,2′,2″,4,4″-pentamethoxytriphenyl carbinol, and combinations thereof. It is further preferable that the pH indicator dye for use with 5AS be selected from the group consisting of phenolphthalein, thymolphthalein, alizarin yellow R, indigo carmine, and combinations thereof. In a most preferred embodiment, the chromogenic substrate is TMB or ABTS and the pH indicator dye is m-cresol purple. In further embodiments of the above kits, the acid stop is modified to inhibit further oxidization of the blue TMB to yellow.

The composition of the present invention can further be provided as a component of peroxidase-linked ELISAs. Therefore, the present invention further includes kits which comprise a peroxidase-linked ELISA for an analyte, one or more first containers each of which contains a mixture of a chromogenic substrate and a pH indicator dye suitable for use with the chromogenic substrate, and one or more second containers each of which contains a peroxide such as hydrogen peroxide or urea peroxide and kits which comprise a peroxidase-linked ELISA for an analyte and one or more containers each of which contains a mixture of a chromogenic substrate, a peroxide such as hydrogen peroxide or urea peroxide, and a pH indicator suitable for use with the chromogenic substrate. In further embodiments of the above kits, the kits further contain a container containing a stop solution.

In the above ELISA kits, it is preferable that the chromogenic substrate is selected from the group consisting of OPD, OND, ABTS, 5AS, and TMB. It is further preferable that the pH indicator dye for acid stops be selected from the group consisting of cresol red, m-cresol purple, benzopurpurin 4B, metanil yellow, 4-phenylazodiphenylamine, malachite green, quinaldine red, orange IV, 2,2′,2″,4,4″-pentamethoxytriphenyl carbinol, and combinations thereof. It is further preferable that the pH indicator dye for use with 5AS be selected from the group consisting of phenolphthalein, thymolphthalein, alizarin yellow R, indigo carmine, and combinations thereof. In a most preferred embodiment, the chromogenic substrate is TMB or ABTS and the pH indicator dye is m-cresol purple. In further embodiments of the above kits, the acid stop is modified to inhibit further oxidization of the blue TMB to yellow.

The above compositions and kits can further include one or more of the visible dyes disclosed in U.S. Pat. No. 6,221,624 B1 to Lihme et al. Thus, the composition of the present invention can include compositions which comprise TMB, pH indicator dye, and a visible dye which is detectable by eye in the reaction vessel of the assay prior to addition of stop solution. The visible dye is selected so as to not substantially interfere with the absorbance of the oxidized TMB or with the oxidation of the TMB and not to substantially interfere with the change in color of the pH indicator dye. Preferably, the above composition further includes hydrogen peroxide. Therefore, in an assay, the visible dye renders the composition visible to the eye when it is added to an immunoassay; the chromogenic substrate enables reactions containing the analyte to be identified; and, the pH indicator dye enables negative reactions to be distinguished from false negative reactions.

The above method and kits can be adapted for use in alkaline phosphatase-based immunoassays such as alkaline phosphatase-based ELISAs. In the case of alkaline-phosphatase-based assays, the composition comprises p-nitrophenyl phosphate (PNNP) and a pH indicator dye. PNNP is hydrolyzed by alkaline phosphatase to p-nitrophenyloxide under basic conditions. Under typical reaction conditions the p-nitrophenyloxide is in equilibrium between its colorless benzenoid form and its yellow quinonoid form. Addition of a stop solution comprising a base such as sodium hydroxide (NaOH) or dipotassium phosphate (K₂HPO₄) to the reaction stops the reaction and shifts the equilibrium towards the quinonoid form. The quinonoid form has a maximum absorbance at 405 and 605 nm. Thus, suitable pH indicator dye for the above reaction should be a dye which changes color at a high pH. Such pH indicator dyes include, but are not limited to, alizarin Yellow R, indigo carmine, and combinations thereof. In the case of alkaline phosphatase-based immunoassays such as alkaline phosphatase-based ELISAs, the pH indicator dye comprising the composition can have a color which is detectable by eye when the composition is applied to the reaction vessel. The pH indicator dye does not need to be colorless or have a color which is essentially not detectable by eye when the composition is applied to the reaction vessel. However, the pH indicator dye comprising the composition must form a second color which is detectable by eye and has an absorbance at a second wavelength when a stop solution is added to the composition in the reaction vessel. The second wavelength cannot substantially interfere with detection of the first wavelength.

The above composition can be provided in one or more containers in a kit. The kit can further include a container containing a stop solution. The composition can further be included as a component of an alkaline phosphatase-linked ELISA kit for detecting particular analytes.

In particular embodiments of the above methods and kits, the composition can further include one or more of the dyes disclosed in U.S. Pat. No. 6,221,624 B1 to Lihme et al.

### EXAMPLE 1

This example shows that in an HRP reaction the absorbance of the m-cresol purple does not interfere with the absorbance of TMB.

The TMB was provided as a 100% water-based solution available as K-BLUE AQUEOUS, Neogen Corporation, Lansing, Mich. To 10 mL of the TMB solution, 22 μg of m-cresol purple was added to make a positive/negative indicator solution. While the m-cresol purple was immediately soluble, to ensure even dispersal of the pH indicator dye, the mixture stirred for about five minutes. The amount of m-cresol purple added was an amount which was determined not to be visually detectable by eye. During stirring, the solution went from colorless to slight straw color. This change in color might have been because of the particular formulation of TMB used. Some TMB formulations will produce a color in solution. It was found to be preferably to add 11 μg of m-cresol purple to 10 mL of the TMB solution instead of 22 μg. At this concentration, the straw-yellow color was essentially indistinguishable to the eye but still produced a detectable rose-pink color upon addition of the stop solution. The performance of the solution containing m-cresol purple at a 2.2 μg/mL concentration was evaluated by incubating a 15 μL aliquot of the solution with a HRP in a quartz cuvette at room temperature. The spectra of the reaction was measured against water on a Shumadzu UV-1601 spectrophotometer.

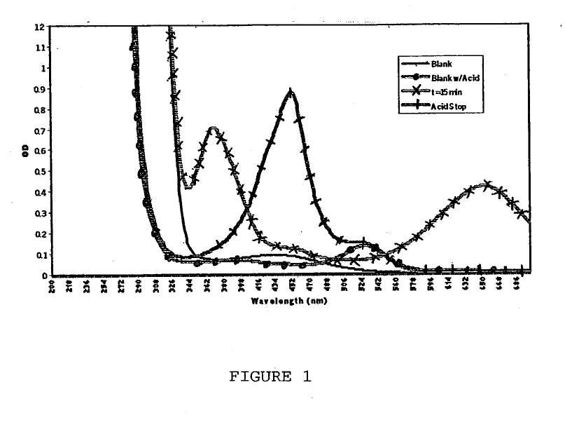

FIG. 1 shows the spectra of the reaction. The black line (-) is the spectra for a blank containing the solution but no HRP and the green line (-·-) is the spectra for the blank with acid added to it. As can be seen the blank was transparent between about 344 nm to greater than 686 nm. Visually, the solution was a light straw color, however, the color was attributable to the TMB formulation. However, adding acid to the solution oxidized the m-cresol purple which produced an absorbance at between about 524 to 542 nm. Visually, the solution was rose-pink. For the cuvette containing HRP, after 15 minutes, the TMB was oxidized to a blue color with a maximum absorbance at about 650 nm (blue line (-×-)). Note that there was no absorbance between about 524 to 542 nm. After adding acid to the reaction, the TMB was further oxidized to its diamine state which had a maximum absorbance at about 450 nm and the m-cresol was oxidized to produce an absorbance at between about 524 to 542 nm (red line (-|-)). Visually, the solution was a yellow and rose-pink blend.

### EXAMPLE 2

In this example, performance of TMB with m-cresol purple in HRP reactions was compared to the performance of TMB alone. In this example, a direct competitive ELISA assay microwell format for detecting opiates was used (Opiate Group 100619 kit from Neogen Corp.). The assay operates on the basis of competition between opiates in the sample and opiate-HRP conjugates for a limited number of specific binding sites in precoated wells of microplates.

In this example, a 180 μL solution of opiate-HRP conjugate was added to each of the precoated wells of a microtiter plate to bind the opiate-HRP to the sites in the wells at room temperature. A set of sixteen wells was reserved to serve as controls. The controls were incubated with buffer blanks which did not contain opiate-HRP. After 45 minutes, the solution was removed from each of the wells and the wells washed three times with 300 μL of wash solution containing phosphate buffered saline and TWEEN. Next, 150 μL of a TMB solution (K-BLUE AQUEOUS, lot No. 020524)) was added to half of the microwells and 150 μL of TMB solution with m-cresol purple at 2.2 μg/mL was added to the other half of the wells. As the reaction progressed, the wells with HRP turned blue. After about 30 minutes, the absorbance at 650 nm for the wells was read using an ELISA reader.

The results from an eight-point standard curve for the wells which had been incubated with opiate-HRP showed that the color absorbance at 650 nm was similar for both sets of wells (1.244 OD for the wells with TMB and 1.201 OD for the wells with TMB with m-cresol purple) and the sensitivities were similar (I-50s of 0.16 ng/mL for the wells with TMB and 0.15 ng/mL for the wells with TMB with m-cresol purple). For the control wells, the OD₆₅₀ was 0.044 for the wells containing TMB and 0.047 for the wells containing TMB with m-cresol blue.

All of the above assays were then stopped by adding 50 μL of 1N HCL to each of the wells. All the wells which had been incubated with opiate-HRP developed a similar color at 450 nm. Acid added to eight of the control wells containing TMB resulted in colorless wells and acid added to the remaining eight control wells containing TMB and m-cresol purple resulted in light rose-pink wells. The absorbance of the rose-pink wells was measured at 562 nm because of the limited filter choices available with the ELISA reader. A 540 nm filter is preferred. The OD₄₅₀ was similar for both sets of controls (0.065 for the control wells containing TMB vs 0.079 for the control wells containing TMB with m-cresol purple. However, the OD₅₆₂ was higher for the control wells containing TMB with m-cresol purple (0.069 for the control wells containing TMB with m-cresol purple vs 0.041 for the control wells containing TMB). The OD₅₆₂ remained relatively stable even 4 hours after the acid had been added (0.079 for the control wells containing TMB with m-cresol purple vs 0.043 for the control wells containing TMB).

In addition to the above, the TMB and the TMB with m-cresol purple compositions were compared by adding two different volumes of each to 4 empty microwells on a standard Costar microtiter plate. The absorbance was measured on an ELISA plate reader using an air blank.

In the first experiment, 100 μL of each composition was added to the empty wells. The average OD₄₅₀ was 0.043 for the empty wells containing TMB and 0.063 for the empty wells containing TMB with m-cresol purple. The average OD₅₆₂ was 0.041 for the empty wells containing TMB and 0.037 for the empty wells containing TMB with m-cresol purple.

In the second experiment, 150 μL of each composition was added to the empty wells. The average OD₄₅₀ was 0.044 for the empty wells containing TMB and 0.078 for the empty wells containing TMB with m-cresol purple. The average OD₅₆₂ was 0.041 for the empty wells containing TMB and 0.040 for the empty wells containing TMB with m-cresol purple. The results show that the m-cresol purple is transparent at the indicator wavelength (OD₅₆₂) in the absence of acid.

### EXAMPLE 3

This example shows that using the m-cresol purple at a concentration of 1.1 μg/mL TMB substrate produced a rose-pink color upon addition of the stop solution even though the color of the solution was indistinguishable before hand.

The TMB solutions used were K-BLUE AQUEOUS, K-BLUE ENHANCED, and K-BLUE MAX (Neogen Corporation, Lansing, Mich.). To a well of a standard COSTAR microplate, 100 μL of TMB solution with or without 1.1 μg/mL m-cresol purple was added. Absorbances were read at 450 nm, 650 nm, and 540 nm. Afterwards, 100 μL of acid stop solution was added and the absorbances were read at 450 nm, 650 nm, and 540 nm. The optical densities were measured on a VMAX microplate reader (Molecular Devices Corporation, Sunnyvale, Calif.) fitted with 450 nm, 650 nm, and 540 nm filters. The blank was air. The results are shown in Table 1.

TABLE 1

| | | | | +100 μL acid | | |
|---|---|---|---|---|---|---|
| | 100 μL substrate | | | (200 μL total) | | |
| Sample | OD₄₅₀ | OD₆₅₀ | OD₅₄₀ | OD₄₅₀ | OD₆₅₀ | OD₅₄₀ |
| K-BLUE | | | | | | |
| AQUEOUS | | | | | | |
| Substrate | 0.044 | 0.037 | 0.039 | 0.040 | 0.035 | 0.036 |
| Substrate w/ | 0.048 | 0.032 | 0.034 | 0.042 | 0.034 | 0.057 |
| indicator | | | | | | |
| K-BLUE | | | | | | |
| ENHANCED | | | | | | |
| Substrate | 0.040 | 0.034 | 0.035 | 0.042 | 0.035 | 0.038 |
| Substrate w/ | 0.052 | 0.036 | 0.037 | 0.042 | 0.032 | 0.057 |
| indicator | | | | | | |
| K-BLUE MAX | | | | | | |
| Substrate | 0.039 | 0.032 | 0.034 | 0.045 | 0.031 | 0.034 |
| Substrate w/ | 0.049 | 0.032 | 0.035 | 0.042 | 0.030 | 0.055 |
| indicator | | | | | | |

For the volumes used, the OD₄₅₀ differences between substrate and substrate with indicator was not readily distinguishable to the eye even though the OD₅₄₀ differences were easily distinguishable to the eye as a light rose-pink color. Thus, this example shows that using the m-cresol purple at a concentration of about 1.1 μg/mL of substrate solution was able to produce a visible color and a detectable absorbance increase at 540 nm after the addition of acid even though a color was not distinguishable before the acid was added.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

We claim:

1. A method for detecting an analyte in a peroxidase-based enzyme-linked immunosorbent assay (ELISA) and distinguishing a negative reaction from a false negative reaction in the ELISA, which comprises:
(a) providing a composition comprising a mixture of a chromogenic substrate which is oxidizable by peroxide in a reaction for the ELISA catalyzed by the peroxidase of the ELISA to form a first color with an absorbance at a first wavelength, a pH indicator dye which forms a second color which is detectable by eye and has an absorbance at a second wavelength when a stop solution is added to the composition in the reaction vessel, and a peroxide;
(b) adding an aliquot of the composition to each of the reaction vessels for the ELISA to form a reaction mixture;
(c) incubating the reaction mixture for a time sufficient to oxidize the chromogenic substrate to the first color;
(d) adding the stop solution to the reaction mixture to stop the reaction and to generate the second color; and
(e) measuring spectrographically absorbances at the first and second wavelengths, wherein absorbance at the first wavelength indicates the detection of the analyte, absorbance at only the second wavelength indicates the negative reaction, and absence of absorbance at the first and second wavelengths indicates the false negative reaction or detecting the second color by eye wherein the second color indicates that the reaction mixture contains the composition and the stop solution.

2. The method of claim 1 wherein the pH indicator is colorless or has a color which is essentially not detectable by eye when the composition is applied to a reaction vessel for the ELISA but which forms the second color which is detectable by eye and has the absorbance at the second wavelength when the stop solution is added to the composition in the reaction vessel.

3. The method of claim 1 wherein the stop solution is an acid and the chromogenic substrate is selected from the group consisting of ortho-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), and 3,3′,5,5′-tetramethylbenzidine (TMB).

4. The method of claim 1 wherein the stop solution is an acid and the chromogenic substrate is 2,2′-azinobis-(3-ethyl-benzothiazoline-6-sulfonic acid) (ABTS).

5. The method of claim 1 wherein the pH indicator dye is selected from the group consisting of cresol red, m-cresol purple, metanil yellow, 4-phenylazodiphenylamine, malachite green, orange IV, 2,2′,2″,4,4″-pentamethoxytriphenyl carbinol, and combinations thereof.

6. The method of claim 1 wherein the stop solution is a base and the chromogenic substrate is 5-aminosalicylic acid (5AS).

7. The method of claim 6 wherein the pH indicator dye is selected from the group consisting of phenolphthalein, thymolphthalein, alizarin Yellow R, indigo carmine, and combinations thereof.

8. The method of claim 1 wherein the ELISA is selected from the group consisting of direct, indirect, and competitive ELISAs.

9. A method for detecting an analyte using 3,3′,5,5′-tetramethylbenzidine (TMB) as a chromogenic substrate in an enzyme-linked immunosorbent assay (ELISA) and distinguishing a negative reaction from a false negative reaction in the ELISA, which comprises:
(a) providing a composition comprising a mixture of the TMB which is oxidizable by peroxide in a reaction for the ELISA catalyzed by the peroxidase of the ELISA to form a first color with an absorbance at a first wavelength, a pH indicator dye which is colorless or has a color which is essentially not detectable by eye when the composition is applied to a reaction vessel for the ELISA but which forms a second color which is detectable by eye and has an absorbance at a second wavelength when a stop solution is added to the composition in the reaction vessel, and a peroxide;
(b) adding an aliquot of the composition to each of the reaction vessels for the ELISA to form a reaction mixture;
(c) incubating the reaction mixture for a time sufficient to oxidize the TMB to the first color;
(d) adding the stop solution to the reaction mixture to stop the reaction, which produces the second color and further oxidizes the TMB to the first color; and
(e) measuring spectrographically absorbances at the first and second wavelengths, wherein absorbance at the first wavelength indicates the detection of the analyte, absorbance at only the second wavelength indicates the negative reaction, and absence of absorbance at the first and second wavelengths indicates the false negative reaction or detecting the second color by eye wherein the second color indicates that the reaction mixture contains the composition and the stop solution.

10. The method of claim 9 wherein the pH indicator dye is selected from the group consisting of cresol red, m-cresol purple, metanil yellow, 4-phenylazodiphenylamine, malachite green, orange IV, 2,2′,2″,4,41″-pentamethoxytriphenyl carbinol, and combinations thereof.

11. The method of claim 9 wherein the pH indicator dye is m-cresol purple.

12. The method of claim 9 wherein the first wavelength is about 450 nm and the second wavelength is over 500 nm.

13. The method of claim 9 wherein the ELISA is selected from the group consisting of direct, indirect, and competitive ELISAs.

14. The method of claim 9 wherein the acid is selected from the group consisting of HCL, H₂SO₄, and H₂PO₄.

15. An enzyme immunoassay for colorimetric detection of an analyte of a type wherein a quantity of a first antibody is adsorbed to a solid support in a reaction vessel; a conjugate is formed between an immunologic reagent and a peroxidase; the conjugate is admixed with a sample to be tested for the analyte, the analyte binds to the first antibody and to the conjugate to form an immunologic complex in solid phase; and the quantity of the analyte is determined by measuring the reaction of the immunologic complex with a chromogenic substrate oxidizable by peroxide and the peroxidase, wherein the improvement comprises:
(a) providing a liquid solution comprising the chromogenic substrate, peroxide, and a pH indicator dye, wherein the chromogenic substrate is oxidizable to a first color with an absorbance at a first wavelength and the pH indicator dye produces a second color which is detectable by eye and which has an absorbance at a second wavelength when a stop solution is added to the reaction vessel containing the liquid solution;
(b) mixing the liquid solution with the immunologic complex in the reaction vessel to form a reaction mixture which oxidizes the chromogenic substrate;
(c) adding the stop solution to the reaction mixture in the reaction vessel to produce the first and second colors; and,
(d) measuring spectrographically the absorbances at the first and second wavelengths, wherein absorbance at the first wavelength indicates detection of the analyte, absorbance at the second wavelength indicates a negative reaction, and an absence of absorbance at the first or second wavelength indicates a false negative reaction or detecting the second color by eye wherein the second color indicates that the reaction mixture contains the liquid solution and the stop solution.

16. The method of claim 15 wherein the pH indicator is colorless or has a color which is essentially not detectable by eye when the composition is applied to a reaction vessel for the ELISA but which forms the second color which is detectable by eye and has the absorbance at the second wavelength when the stop solution is added to the composition in the reaction vessel.

17. The method of claim 15 wherein the stop solution is an acid and the chromogenic substrate is selected from the group consisting of ortho-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), and 3,3′,5,5′-tetramethylbenzidine (TMB).

18. The method of claim 15 wherein the stop solution is an acid and the chromogenic substrate is 2,2′-azinobis-(3-ethyl-benzothiazoline-6-sulfonic acid) (ABTS).

19. The method of claim 15 wherein the pH indicator dye is selected from the group consisting of cresol red, m-cresol purple, metanil yellow, 4-phenylazodiphenylamine, malachite green, orange IV, 2,2′,2″,4,4″-pentamethoxytriphenyl carbinol, and combinations thereof.

20. The method of claim 15 wherein the stop solution is a base and the chromogenic substrate is 5-aminosalicylic acid (5AS).

21. The method of claim 20 wherein the pH indicator dye is selected from the group consisting of phenolphthalein, thymolphthalein, alizarin Yellow R, indigo carmine, and combinations thereof.

22. An enzyme immunoassay for colorimetric detection of an antibody of a type wherein a quantity of an analyte is adsorbed to a solid support in a reaction vessel; a conjugate is formed between an immunologic reagent and a peroxidase; the conjugate is admixed with a sample to be tested for the antibody, the antibody binds to the analyte and to the conjugate to form an immunologic complex in solid phase; and the quantity of the antibody is determined by measuring the reaction of the immunologic complex with a chromogenic substrate oxidizable by peroxide and the peroxidase, wherein the improvement comprises:
(a) providing a liquid solution comprising the chromogenic substrate, peroxide, and a pH indicator dye, wherein the chromogenic substrate is oxidizable to a first color with an absorbance at a first wavelength and the pH indicator dye produces a second color which is detectable by eye and which has an absorbance at a second wavelength when a stop solution is added to the reaction vessel containing the liquid solution;
(b) mixing the liquid solution with the immunologic complex in the reaction vessel to form a reaction mixture which oxidizes the chromogenic substrate;
(c) adding the stop solution to the reaction mixture in the reaction vessel to produce the first and second colors; and,
(d) measuring spectrographically the absorbances at the first and second wavelengths, wherein absorbance at the first wavelength indicates detection of the analyte, absorbance at the second wavelength indicates a negative reaction, and an absence of absorbance at the first or second wavelength indicates a false negative reaction or detecting the second color by eye wherein the second color indicates that the reaction mixture contains the liquid solution and the stop solution.

23. The method of claim 22 wherein the pH indicator is colorless or has a color which is essentially not detectable by eye when the composition is applied to a reaction vessel for the ELISA but which forms the second color which is detectable by eye and has the absorbance at the second wavelength when the stop solution is added to the composition in the reaction vessel.

24. The method of claim 22 wherein the stop solution is an acid and the chromogenic substrate is selected from the group consisting of ortho-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), and 3,3′,5,5′-tetramethylbenzidine (TMB).

25. The method of claim 22 wherein the stop solution is an acid and the chromogenic substrate is 2,2′-azinobis-(3-ethyl-benzothiazoline-6-sulfonic acid) (ABTS).

26. The method of claim 22 wherein the pH indicator dye is selected from the group consisting of cresol red, m-cresol purple, metanil yellow, 4-phenylazodiphenylamine, malachite green, orange IV, 2,2′,2″,4,4″-pentamethoxytriphenyl carbinol, and combinations thereof.

27. The method of claim 22 wherein the stop solution is a base and the chromogenic substrate is 5-aminosalicylic acid (5AS).

28. The method of claim 22 wherein the pH indicator dye is selected from the group consisting of phenolphthalein, thymolphthalein, alizarin Yellow R, indigo carmine, and combinations thereof.

29. An enzyme immunoassay for colorimetric detection of an analyte of a type wherein a quantity of a first antibody is adsorbed to a solid support in a reaction vessel; a conjugate is formed between an immunologic reagent and a peroxidase; the conjugate is admixed with a sample to be tested for the analyte, the analyte binds to the first antibody and to the conjugate to form an immunologic complex in solid phase; and the quantity of the analyte is determined by measuring the reaction of the immunologic complex with 3,3′,5,5′-tetramethylbenzidine (TMB) which is oxidizable by peroxide and the peroxidase, wherein the improvement comprises:
(a) providing a liquid solution comprising the TMB, peroxide, and a pH indicator dye, wherein the TMB is oxidizable to a first color with an absorbance at a first wavelength and the pH indicator dye is colorless or has a color which is essentially not detectable by eye when the liquid solution is applied to the reaction vessel for the immunoassay but which produces a second color which is detectable by eye and which has an absorbance at a second wavelength when a stop solution is added to the reaction vessel containing the liquid solution;
(b) mixing the liquid solution with the immunologic complex in the reaction vessel to form a reaction mixture which oxidizes the TMB;
(c) adding the stop solution to the reaction mixture in the reaction vessel to produce the first and second colors; and,
(d) measuring spectrographically the absorbances at the first and second wavelengths, wherein absorbance at the first wavelength indicates detection of the analyte, absorbance at the second wavelength indicates a negative reaction, and an absence of absorbance at the first or second wavelength indicates a false negative reaction or detecting the second color by eye wherein the second color indicates that the reaction mixture contains the liquid solution and the stop solution.

30. The method of claim 29 wherein the pH indicator dye is selected from the group consisting of cresol red, m-cresol purple, metanil yellow, 4-phenylazodiphenylamine, malachite green, orange IV, 2,2′,2″,4,4″-pentamethoxytriphenyl carbinol, and combinations thereof.

31. The method of claim 29 wherein the pH indicator dye is m-cresol purple.

32. The method of claim 29 wherein the first wavelength is about 450 nm and the second wavelength is over 500 nm.

33. The method of claim 29 wherein the acid is selected from the group consisting of HCL, H₂SO₄, and H₂PO₄.

34. An enzyme immunoassay for colorimetric detection of an antibody of a type wherein a quantity of an analyte is adsorbed to a solid support in a reaction vessel; a conjugate is formed between an immunologic reagent such as an antibody, Fab fragment, Fv fragment, single-chain Fv polypeptide, or other antibody derivative and a peroxidase; the conjugate is admixed with a sample to be tested for the antibody, the antibody binds to the analyte and to the conjugate to form an immunologic complex in solid phase; and the quantity of the antibody is determined by measuring the reaction of the immunologic complex with 3,3′,5,5′-tetramethylbenzidine (TMB) which is oxidizable by peroxide and the peroxidase, wherein the improvement comprises:
(a) providing a liquid solution comprising the TMB, peroxide, and a pH indicator dye, wherein the TMB is oxidizable to a first color with an absorbance at a first wavelength and the pH indicator dye is colorless or has a color which is essentially not detectable by eye when the liquid solution is applied to the reaction vessel for the immunoassay but which produces a second color which is detectable by eye and which has an absorbance at a second wavelength when a stop solution is added to the reaction vessel containing the liquid solution;
(b) mixing the liquid solution with the immunologic complex in the reaction vessel to form a reaction mixture which oxidizes the TMB;
(c) adding the stop solution to the reaction mixture in the reaction vessel to produce the first and second colors; and,
(d) measuring spectrographically the absorbances at the first and second wavelengths, wherein absorbance at the first wavelength indicates detection of the analyte, absorbance at the second wavelength indicates a negative reaction, and an absence of absorbance at the first or second wavelength indicates a false negative reaction or detecting the second color by eye wherein the second color indicates that the reaction mixture contains the liquid solution and the stop solution.

35. The method of claim 34 wherein the pH indicator dye is selected from the group consisting of cresol red, m-cresol purple, metanil yellow, 4-phenylazodiphenylamine, malachite green, orange IV, 2,2′,2″,4,4″-pentamethoxytriphenyl carbinol, and combinations thereof.

36. The method of claim 34 wherein the pH indicator dye is m-cresol purple.

37. The method of claim 34 wherein the first wavelength is about 450 nm and the second wavelength is over 500 nm.

38. The method of claim 34 wherein the acid is selected from the group consisting of HCL, H₂SO₄, and H₂PO₄.

39. A kit for detecting an analyte in an enzyme-linked assay and distinguishing a negative reaction from a false negative reaction in the assay, which comprises:
one or more first containers each of which contains a mixture of a chromogenic substrate, a pH indicator dye suitable for use with the chromogenic substrate, which produces a color detectable by eye when an acid or base stop solution is added to the mixture in the reaction vessel, and a peroxide.

40. The kit of claim 39 wherein the pH indicator is colorless or has a color which is essentially not detectable by eye when the composition is applied to a reaction vessel for the assay but which forms the second color which is detectable by eye and has the absorbance at the second wavelength when the stop solution is added to the composition in the reaction vessel.

41. The kit of claim 39 wherein the first container contains the chromogenic substrate and the pH indicator dye and the peroxide is contained in a second container.

42. The kit of claim 39 or 40 wherein the kit further includes a third container containing an acid or base stop.

43. The kit of claim 39 wherein the chromogenic substrate is selected from the group consisting of ortho-phenylenediamine (OPD), 2,2′-azinobis-(3-ethyl-benzothiazoline-6-sulfonic acid) (ABTS), diaminobenzidine (DAB), 3,3′dimethyloxybenzidine (ortho-dianisidine or ODN), and 3,3′ 5,5′-tetramethylbenzidine (TMB).

44. The kit of claim 39 wherein the pH indicator dye is selected from the group consisting of cresol red, m-cresol purple, metanil yellow, 4-phenylazodiphenylamine, malachite green, quinaldine red, orange IV, 2,2′,2″,4,4″-pentamethoxytriphenyl carbinol, and combinations thereof.

45. The kit of claim 39 wherein the chromogenic substrate is 5-aminosalicylic acid (5AS).

46. The kit of claim 45 wherein the pH indicator dye is selected from the group consisting of phenolphthalein, thymolphthalein, alizarin yellow R, indigo carmine, and combinations thereof.

47. The kit of claim 39 wherein the pH indicator dye is m-cresol purple and the chromogenic substrate is selected from the group consisting of 3,3′,5,5′-tetramethylbenzidine (TMB) and 2,2′-azinobis-(3-ethyl-benzothiazoline-6-sulfonic acid) (ABTS).

48. The kit of claim 39 wherein the enzyme-linked assay is an enzyme-linked immunosorbent assay (ELISA).

49. A composition for use in an enzyme-linked immunosorbent assay (ELISA) and which enables a negative reaction to be distinguished from a false negative reaction in the ELISA, which comprises in a mixture:
(a) a chromogenic substrate which is oxidizable by a peroxide generated in the ELISA in a reaction mixture to which a stop solution is then added to stop the reaction to form a first color with an absorbance at a first wavelength; and
(b) a pH indicator dye which is colorless or has a color which is essentially not detectable by eye when the composition is applied to a reaction vessel for the ELISA but which produces a second color detectable by eye and which has an absorbance at a second wavelength when an acid or base stop solution is added to the composition in the reaction vessel.

50. The composition of claim 49 wherein the chromogenic substrate is selected from the group consisting of ortho-phenylenediamine (OPD), 2,2′-azinobis-(3-ethyl-benzothiazoline-6-sulfonic acid) (ABTS), diaminobenzidine (DAB), and 3,3′dimethyloxybenzidine (ortho-dianisidine or ODN).

51. The composition of claim 50 wherein the pH indicator dye is selected from the group consisting of cresol red, m-cresol purple, metanil yellow, 4-phenylazodiphenylamine, malachite green, quinaldine red, orange IV, 2,2′,2″,4,4″-pentamethoxytriphenyl carbinol, and combinations thereof.

52. The composition of claim 49 wherein the chromogenic substrate is 5-aminosalicylic acid (5AS).

53. The composition of claim 52 wherein the pH indicator dye is selected from the group consisting of phenolphthalein, thymolphthalein, alizarin yellow R, indigo carmine, and combinations thereof.

54. The composition of claim 49 wherein the pH indicator dye is m-cresol purple and the chromogenic substrate is 2,2′-azinobis-(3-ethyl-benzothiazoline-6-sulfonic acid) (ABTS).

55. A composition for use in an enzyme-linked immunosorbent assay (ELISA) and which enables a negative reaction to be distinguished from a false negative reaction in the ELISA, which comprises in an aqueous mixture:
(a) 3,3′,5,5′-tetramethylbenzidine (TMB) which is oxidizable by a peroxide generated in the ELISA in a reaction mixture to which a stop solution is then added to stop the reaction to form a first color with an absorbance at a first wavelength; and
(b) a pH indicator dye which is colorless or has a color which is essentially not detectable by eye when the composition is applied to a reaction vessel for the ELISA but which produces a second color detectable by eye and which has an absorbance at a second wavelength when an acid or base stop solution is added to the composition in the reaction vessel.

56. The composition of claim 55 wherein the pH indicator dye is selected from the group consisting of cresol red, metanil yellow, 4-phenylazodiphenylamine, malachite green, orange IV, 2,2′,2″,4,4″-pentamethoxytriphenyl carbinol, and combinations thereof.

57. The composition of claim 55 wherein the pH indicator dye is m-cresol purple.

58. A method for detecting an analyte in a enzyme-linked assay and distinguishing a negative reaction from a false negative reaction in the assay, which comprises:
(a) providing a composition comprising a mixture of a chromogenic substrate selected from the group consisting of ortho-phenylenediamine (OPD), 2,2′-azinobis-(3-ethyl-benzothiazoline-6-sulfonic acid) (ABTS), diaminobenzidine (DAB), and 3,3′dimethyloxybenzidine (ortho-dianisidine or ODN) which is converted by an enzyme in a reaction vessel for the assay to a first color which has an absorbance at a first wavelength and a pH indicator dye which produces a second color detectable by eye and which has an absorbance at a second wavelength when an acid or base stop solution is added to the composition in the reaction vessel;
(b) adding an aliquot of the composition to the reaction vessel for the enzyme-linked assay to form a reaction mixture;
(c) incubating the reaction mixture for a time sufficient to produce the first color;
(d) adding the stop solution to the reaction mixture to stop the reaction and to generate the second color with the second wavelength; and
(e) measuring spectrographically absorbances at the first and second wavelengths, wherein absorbance at the first wavelength indicates the detection of the analyte, absorbance at only the second wavelength indicates the negative reaction, and absence of absorbance at the first and second wavelengths indicates the false negative reaction or detecting the second color by eye wherein the second color indicates that the reaction mixture contains the composition and the stop solution.

59. The method of claim 58 wherein the enzyme is an alkaline phosphatase, the chromogenic substrate is p-nitrophenyl phosphate, and the stop solution is a base.

60. The method of claim 59 wherein the pH indicator dye is selected from the group consisting of alizarin Yellow R, indigo carmine, and combinations thereof.

61. The method of claim 58 wherein the pH indicator dye is selected from the group consisting of cresol red, m-cresol purple, metanil yellow, 4-phenylazodiphenylamine, malachite green, orange IV, 2,2′,2″,4,4″-pentamethoxytriphenyl carbinol, and combinations thereof.

62. The method of claim 58 wherein the assay is an enzyme-linked immunosorbent assay (ELISA).

63. The method of claim 58 or 62 wherein the assay is selected from the group consisting of direct, indirect, and competitive assay.

64. A kit for detecting an analyte in an enzyme-linked assay and distinguishing a negative reaction from a false negative reaction in the assay, which comprises:
one or more containers each of which contains a mixture of a chromogenic substrate selected from the group consisting of ortho-phenylenediamine (OPD), 2,2′-azinobis-(3-ethyl-benzothiazoline-6-sulfonic acid) (ABTS), diaminobenzidine (DAB), and 3,3′dimethyloxybenzidine (ortho-dianisidine or ODN) which is converted by an enzyme in a reaction vessel for the assay to a first color and a pH indicator dye suitable for use with the chromogenic substrate which produces a second color detectable by eye when an acid or base stop solution is added to the mixture in the reaction vessel.

65. The kit of claim 64 wherein the chromogenic substrate is p-nitrophenyl phosphate.

66. The kit of claim 65 wherein the pH indicator dye is selected from the group consisting of alizarin Yellow R, indigo carmine, and combinations thereof.

67. The kit of claim 64 wherein the pH indicator dye is selected from the group consisting of cresol red, m-cresol purple, metanil yellow, 4-phenylazodiphenylamine, malachite green, orange IV, 2,2′,2″,4,4″-pentamethoxytriphenyl carbinol, and combinations thereof.

68. The kit of claim 64 wherein the assay is an enzyme-linked immunosorbent assay (ELISA).

69. A composition for use in an enzyme-linked assay and which enables a negative reaction to be distinguished from a false negative reaction in the assay, which comprises in a mixture:
(a) a chromogenic substrate is selected from the group consisting of ortho-phenylenediamine (OPD), 2,2′-azinobis-(3-ethyl-benzothiazoline-6-sulfonic acid) (ABTS), diaminobenzidine (DAB), and 3,3′dimethyloxybenzidine (ortho-dianisidine or ODN) which is converted by an enzyme in a reaction vessel for the assay to a first color; and
(b) a pH indicator dye which produces a second color detectable by eye when an acid or base stop solution is added to the composition in the reaction vessel.

70. The composition of claim 69 wherein the chromogenic substrate is p-nitrophenyl phosphate.

71. The composition of claim 70 wherein the pH indicator dye is selected from the group consisting of alizarin Yellow R, indigo carmine, and combinations thereof.

72. The composition of claim 69 wherein the pH indicator dye is selected from the group consisting of cresol red, m-cresol purple, metanil yellow, 4-phenylazodiphenylamine, malachite green, orange IV, 2,2′,2″,4,4″-pentamethoxytriphenyl carbinol, and combinations thereof.
